(19) United States
(12) Patent Application Publication
(10) Pub. No.: US 2009/0298064 A1
(43) Pub. Date: Dec. 3, 2009

(54) Genomic Sequencing

(76) Inventors: Serafim Batzoglou, Palo Alto, CA (US); Mostafa Ronaghi, Los Altos Hills, CA (US); Andreas Sundquist, Palo Alto, CA (US)

Correspondence Address: CRAWFORD MAUNU PLLC, 1150 NORTHLAND DRIVE, SUITE 100, ST. PAUL, MN, 55120, US

(21) Appl. No.: 12/129,330

(22) Filed: May 29, 2008

(51) Int. Cl. C12Q 1/68

(52) U.S. Cl. 435/6

(57) ABSTRACT

Genomic sequencing is implemented for high throughput applications that can include short reads. In one example, whole-genome sequencing involves a method in which a subset of fragments of a target genome are selected as a random function, and each fragment is replicated into clones. The clones are ordered into clone contigs based on sets of overlapping clones, and potential read overlaps are determined from clone read data. The method can also involve reading local assemblies of contigs from regions smaller than a clone length and assembling the local assemblies into read sets, combining the assembled read sets into clone-sized regions and assembling the clone-sized regions, and assembling the clone-sized regions into clone contigs. Overlapping sets of clones and their ordering can be determined computationally from read data, with a high depth of clone coverage to provide a large number of boundaries on which the assemblies can be segmented into overlapping regions of pooled reads.

### RELATED DOCUMENTATION

This patent document includes an Appendix which is incorporated by reference, as discussed herein.

### FIELD OF THE INVENTION

The present invention relates generally to genomic sequencing, systems and methods relating thereto.

### BACKGROUND

Sequencing technology has come a long way since shotgun sequencing and assembly were first introduced as a methodology for sequencing genomes. Initially only applicable to small genomic sequences such as the genome of the bacteriophage 1, viruses and bacterial artificial chromosomes (BACs), sequencing was expensive and required a great deal of manual labor in order to assemble the reads into the underlying sequence.

Today, sequencing and assembly methodologies can be applied to mammalian genomes and most of the labor is automated. Sanger sequencing based on gel electrophoresis, still the dominant sequencing technology, can produce random sequence reads that are between 500 and 1000 base pairs long with less than 1% error rate at a relatively low cost.

Complex genomes contain many repetitive sequences that make it challenging to assemble reads of the sequence into the underlying sequence. To help the process of assembly, reads are obtained with some long-range information. Two common methods are doublebarreled sequencing and hierarchical sequencing. In doublebarreled sequencing, pairs of reads are obtained from both ends of inserts of various sizes. Whole-genome double-barreled shotgun sequencing has been used successfully to assemble several complex genomes, and a number of different assemblers have been developed for this purpose.

In hierarchical sequencing, the genome is covered by cloned inserts such as BACs and reads are obtained separately from each clone. Paired reads can resolve repeats by jumping across them and disambiguating the ordering of flanking unique regions. Hierarchical sequencing relies on clustering reads into small local sets that represent the sequence of one clone, where most of the repeats have a unique copy and therefore assembly is straight-forward. This technique has been used to sequence several genomes including those of the yeast Saccharomyces cerevisiae, Caenorhabditis elegans, and human. Most applications of hierarchical sequencing have been performed under the “map first, sequence second” or physical mapping approach. In the physical mapping approach, a complete physical map of a large set of clones is first constructed, covering the genome with redundancy. Then, a minimal tiling subset of those clones is selected and fully sequenced.

Unfortunately, the cost of sequencing and assembling genomes (e.g., mammalian genomes) is still on the order of tens of millions of dollars and involves months of factory-style sequencing. Many sequencing approaches have attempted to address these costs and timing characteristics, but have been limited in their ability to produce useful and desirable results. For instance, while recent short-read sequencing technologies may enjoy relatively reduced sequencing costs, their limitations have prevented the de novo sequencing of eukaryotic genomes with the standard shotgun sequencing protocol.

These and other issues have been challenging to the sequencing of genomes, and in particular, to rapid, economical sequencing of genomes to produce high-quality assemblies.

### SUMMARY

Various embodiments of the present invention are directed to addressing the above issues and others relating to the sequencing of genomes. Certain exemplary embodiments use an approach that is amenable to relatively fast, economical sequencing of whole genomes and/or directed at applications involving genomic sequencing for high throughput applications that can include short reads. These and other aspects of the present invention are exemplified in a number of implementations and applications, some of which are shown in the figures and characterized in the claims section that follows.

In specific embodiments, the present invention is directed to a whole-genome sequencing method in which a subset of fragments of a target genome are selected as a random function, and each fragment is replicated into clones. The clones are ordered into clone contigs based on sets of overlapping clones, and potential read overlaps are determined from clone read data. The method can also involve reading local assemblies of contigs from regions smaller than a clone length and assembling the local assemblies into read sets, combining the assembled read sets into clone-sized regions and assembling the clone-sized regions, and assembling the clone-sized regions into clone contigs. Overlapping sets of clones and their ordering can be determined computationally from read data, with a high depth of clone coverage to provide a large number of boundaries on which the assemblies can be segmented into overlapping regions of pooled reads.

Various other aspects of the present invention are directed to aspects of the embodiments disclosed herein. These aspects include, for example, media for storing computer executable code which, when loaded and executed by a computer, causes such methods to be performed, and to steps, devices and/or systems used (in whole or in part) in connection with the embodiments disclosed herein.

The above summary is not intended to describe each illustrated embodiment or every implementation of the present invention.

### BRIEF DESCRIPTION OF THE DRAWINGS

The invention may be more completely understood in consideration of the detailed description of various embodiments of the invention that follows in connection with the accompanying drawings in which:

FIG. 1 shows genome sequencing protocol, ordering and error correction steps, according to an example embodiment of the present invention;

FIG. 2 shows contig assembly steps, according to another example embodiment of the present invention;

FIG. 3 shows an approach for implementation of a sequencing protocol and assembly, according to another example embodiment of the present invention;

FIG. 4A-FIG. 4C show approaches for constructing localized read sets in accordance with various example embodiments of the present invention, and in which
FIG. 4A shows the construction of clone read sets,
FIG. 4B shows the construction of intersection read sets, and
FIG. 4C shows the construction of difference read sets;

FIG. 5 shows an approach for the construction of contig sets from read set assemblies, according to another example embodiment of the present invention; and

FIG. 6 shows an approach to merging contigs, according to another example embodiment of the present invention.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

The present invention is directed to genomic sequencing approaches, their uses and systems for the same. These and other aspects of the present invention are exemplified in a number of illustrated implementations and applications, some of which are shown and characterized in the following description and related figures, and further in the claims section that follows.

According to an example embodiment of the present invention, a genome sequencing protocol and assembly approach utilizes high-throughput short-read technologies. A hierarchical sequencing-type approach is carried out as follows. A clone library is randomly selected, clones are sampled from the genome at relatively high coverage, and reads are made from the clones at relatively low coverage. For instance, reads can be obtained from random clones (i.e., rather than using a tiling path) that cover the genome at high redundancy, and each clone is then sampled at relatively low depth with reads. A determination is made as to which clones overlap each other, and the clones are ordered along the genome. Potential read overlaps are determined and error correction is performed on the base pairs of each read.

Assembly is carried out in three stages. First, read sets are formed by determining all reads that overlap any particular clone and performing set operations to produce small regions to assemble. These reads are then assembled into larger contigs. Contig sets are created by collecting the set of output contigs (from the previous stage) that could overlap any particular clone, and assembling the collection. The remaining set of contigs is merged using a scalable assembler that makes use of the clone ordering.

With these approaches, sequencing is carried out without necessarily using relatively time-consuming physical mapping approaches. In many applications, various sequencing and assembly functions are automated and carried out in parallel, using a microprocessor (e.g., a microcomputer) programmed accordingly.

In a more particular example embodiment, a target genome is sequenced as follows. Multiple copies of the target genome are taken and sheared into BAC insert-sized fragments having a mean length of 150 Kb. A subset of the fragments is randomly selected to relatively high coverage (genome-clone coverage level=Cov_{G}) and a tiling path is computed through the fragments after sequencing. Such a high coverage, low depth embodiment may be relative, for example, to traditional hierarchical sequencing in which a high-coverage clone library is constructed from fragments and a subset of the clones is selected to form a tiling path with minimal overlap (e.g., using techniques such as restriction enzyme fingerprinting). Each fragment is then replicated into clones that are uniquely identified. After replication, reads are sampled from each clone to a particular coverage (clone-read coverage level=Cov_{R}), and each read is labeled with its particular clone of origin. Each clone is sequenced to relatively low coverage to limit the amount of over-sampling, and the net sequencing coverage level is Cov_{G}·Cov_{R}.

Clone ordering is carried out in a variety of manners, depending upon the applications. In one embodiment, for each clone, the k-mer content (the set of all sequences of k bases) of its reads are examined and a clone graph is constructed. The edge weights between two clones in the graph are the count of shared, relatively unique k-mers. A greedy contraction algorithm is run on the graph, merging the nodes into ordered lists of the clones. Upon completion, sets of overlapping clones and their relative ordering (called clone contigs) are determined. This clone ordering approach effectively localizes the overlap detection and assembly problem by restricting the set of possible overlaps for reads to those reads within nearby clones.

Overlap detection and error correction are carried out to ensure accuracy of the reads. In one embodiment, read overlaps are compared and detected sequencing errors are corrected. Using the clone contigs, the search for overlapping reads is restricted to a small set of neighboring clones. Transitive overlaps are used to achieve desirable overlap detection sensitivity, and multiple alignments of the reads are created to detect false overlaps, using excessive or correlated errors (a signature of repeated sequence). Errors are corrected using a consensus, including errors in a homopolymeric run count (e.g., similar to Pyrosequencing approaches).

With overlaps identified, contig assembly is carried out. In one embodiment, contig assembly is carried out in three stages on progressively larger regions, as generally described above. Read sets are created in a first stage, with the sets including reads selected from multiple clones contained within sub-regions smaller than a clone length. These read sets are constructed by first finding all reads that overlap each particular clone, and then performing intersection and subtraction operations on the sets of reads to isolate smaller regions. Each read set is then assembled independently (e.g., using a numerical software such as the program Euler available from R. Grothmann, including version 5.0, released in 2008). In the second stage, larger contig sets are created by combining the contigs resulting from the previous stage in larger clone-sized regions for assembly (i.e., also with a numerical software program). In the third stage, a custom assembler is used to assemble clone contigs from the results of the second stage.

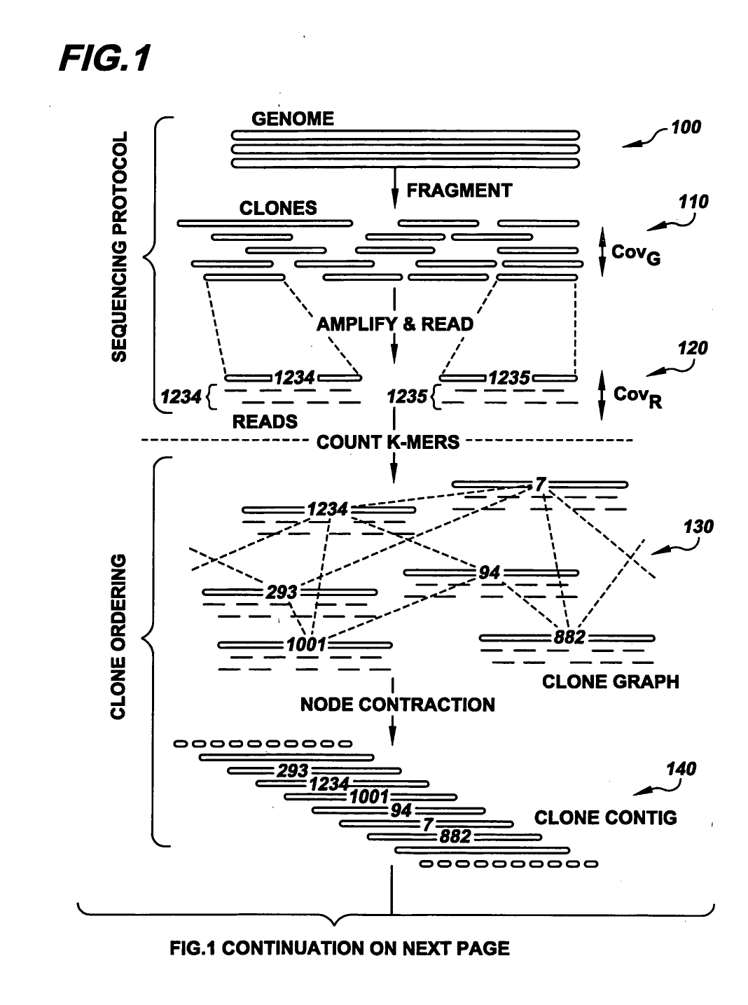

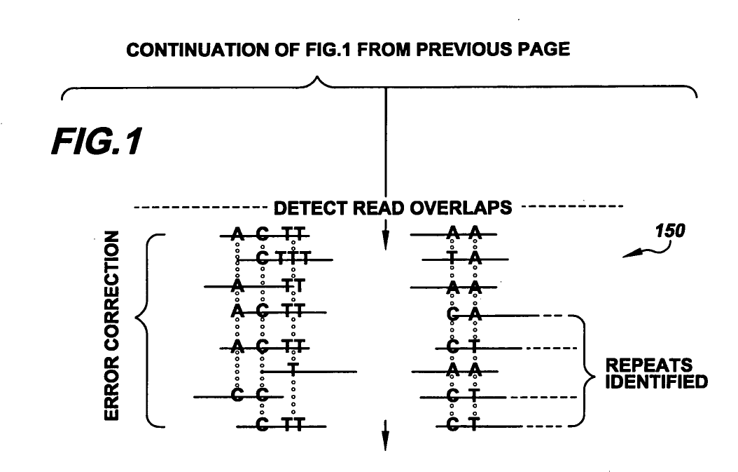

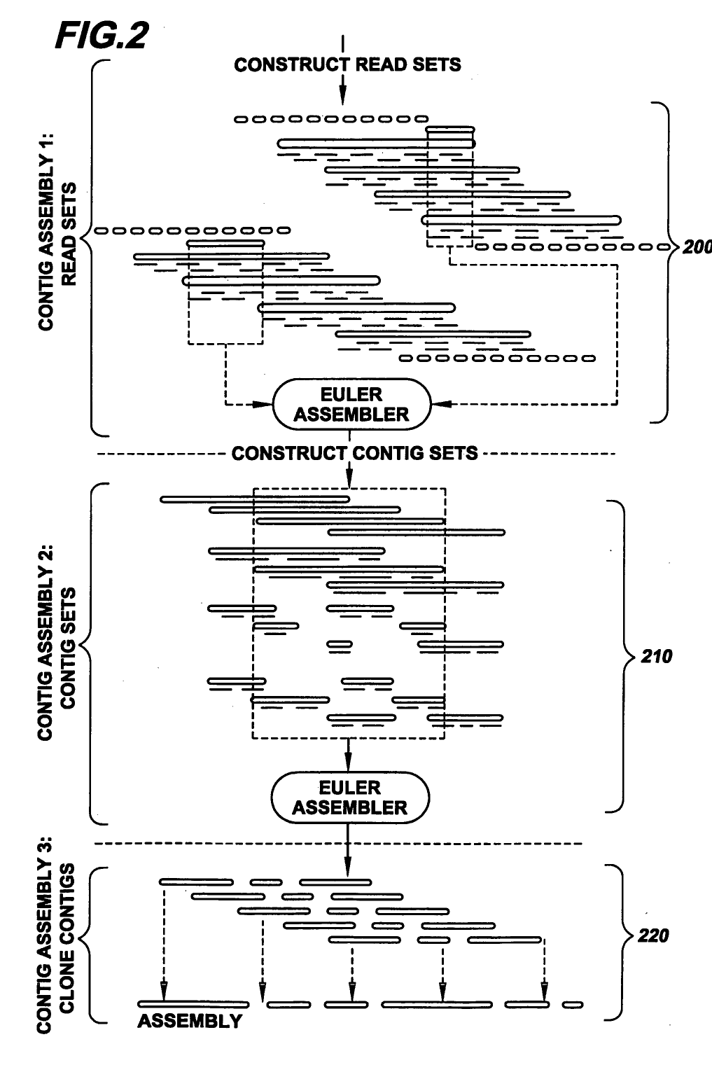

Turning now to the figures, FIG. 1 shows genome sequencing protocol, ordering and error correction steps, according to another example embodiment of the present invention. FIG. 2 shows contig assembly steps, according to another example embodiment of the present invention. For illustrative purposes, FIG. 1 is discussed together with FIG. 2 in connection with a combined sequencing approach. These embodiments are appropriate, for example, for unpaired, short reads in which clones are augmented with reads from other clones.

Referring to FIG. 1, a genome 100 is fragmented as shown at 110 at high coverage (Cov_{G}), and amplified and read at low coverage (Cov_{R}) at 120. At 130, the k-mers are counted and, from the k-mer content of each clone, a clone graph is constructed with edge weights reflecting likely clone proximities. At 140, node contraction is carried out and the clone graph is used by a clone ordering algorithm to determine the clone contigs. For instance, edges of the clone graph can be contracted in order of decreasing weight; after each contraction step, a local optimization procedure is applied to reorder the clones near the junction according to their pairwise edge weights. Putative read overlaps are detected at 150 by looking in nearby clones, and error correction is performed upon overlaps that do not match.

Referring to FIG. 2, three stages of contig assembly are shown. In a first stage 200, read sets are created via set operations that include reads from multiple overlapping clones within small clone sub-regions, with intersection and subtraction read sets shown. The read sets are assembled using a numerical software program (Euler shown by way of example). At a second stage 210, contigs resulting from the first stage are combined in clone-sized contig sets for assembly (Euler again shown by way of example). In a third stage 220, a scalable assembler is used to merge clone contigs including, for example, an entire clone contig.

The approaches shown in FIG. 1 and in FIG. 2 can be carried out in a variety of manners. In one application, clones of the size 150±10 Kb are randomly and uniformly selected from across a genome, reaching a given clone depth of coverage Cov_{G}=7.5x or 10.0x. Next, reads are generated in a similar uniform fashion from each clone with a depth given by the read coverage Cov_{R}=1.5x or 2.0x. Read lengths can vary; for some applications the lengths are 200±25 bp, and in other applications between about 100 bp and 300 bp with a proportional standard deviation used to assess the effect of read length on assembly quality. In many applications, read lengths of 200 bp or higher produce assemblies with desirable contig lengths and misassembly rates. The read length can be increased from about 200 bp to 250 bp to improve sequence coverage, assembled contig sizes, and number of misassemblies. For various embodiments, this range has been determined to be useful, with increasing the read length to 300 bp not yielding (relatively) significant gains. Other embodiments are directed to increasing the coverage level (e.g., from 11.25x to 20.0x) to improve quality at relatively low read lengths (e.g., about 200 bp) and/or without increasing read length.

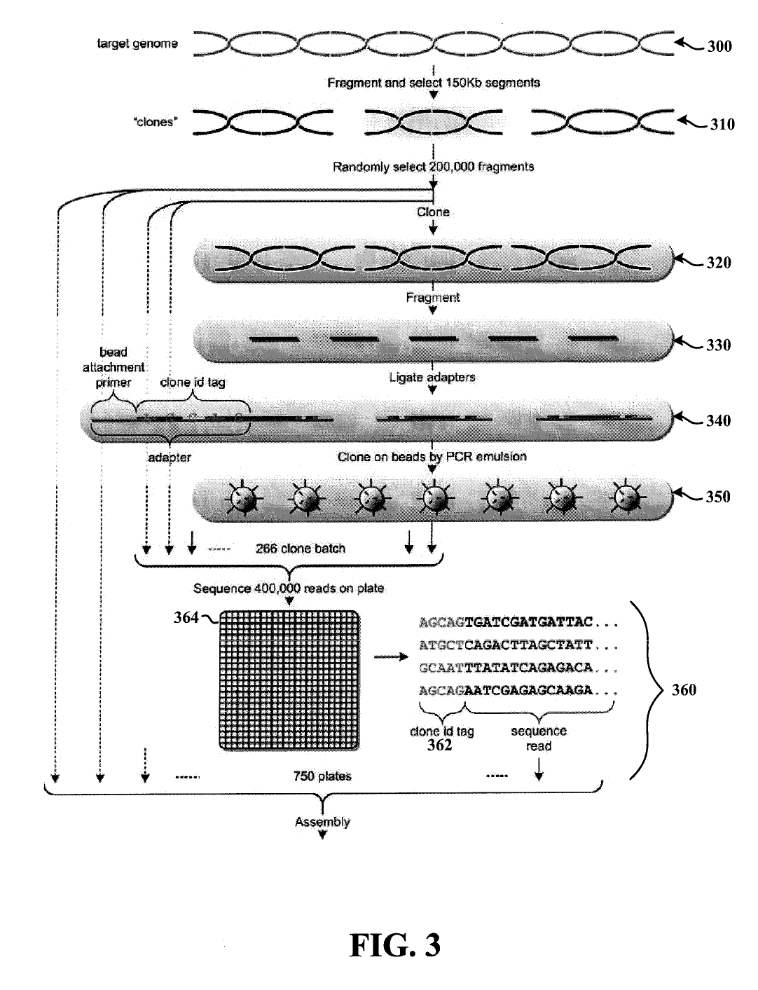

FIG. 3 shows an approach for implementation of a sequencing protocol and assembly, according to a more particular example embodiment of the present invention. A sequencing protocol is executed at 20.0x total coverage for a 3 Gb mammalian genome as follows. DNA from the target organism genome 300 is purified and fragmented, and 150 Kb-sized fragments are isolated at 310. 200,000 fragments are randomly picked and cloned individually at 320. Generally, there is no need to label and store the clones beyond the ability to distinguish between the 266 clones in each “batch” described in the following.

Since each 454 sequencing plate (using technology commercially available from 454 Life Sciences of Branford, Conn.) can perform 250,000 reads, 266 clones are multiplexed on each 400,000 read plate to read 200 bp fragments at 2.0x coverage from each clone. Before mixing together the batch, the clones are fragmented at 330, and adapters containing the bead attachment primer along with a unique 5-base tag are ligated at 340. At 350, the read fragments are amplified on beads using PCR emulsion. As shown at 360, the first 5 bases (at 362) of each read are used to identify the clone within the batch from which it was sequenced. By running 750 plates in this fashion, a mammalian genome can be fully sequenced to 20.0x coverage.

In some embodiments, a scaffolding approach is carried out as a post-processing step to order and orient the contigs in scaffolds. Paired reads are very lightly sequenced using an ultra high-throughput sequencing technology such as Polony sequencing (see, e.g., Shendure J, Porreca G J, Reppas N B, Lin X, McCutcheon J P, et al. (2005) Accurate Multiplex Polony Sequencing of an Evolved Bacterial Genome, Science 309: 1728-1732), or Sequencing-By-Synthesis technology from Solexa. Although reads may be as short as 25 bp, the majority of these are unique in the genome up to two base differences, allowing them to serve as anchors to link together contigs. After indexing the assembly contigs, the paired reads are filtered for those that anchor uniquely in the assembly, a minimum threshold of 5 paired read links is used to join two contigs into a scaffold.

Referring back to FIG. 1, and as may be applicable to FIG. 3, clone ordering at 130 and 140, and subsequent overlap detection/error correction at 150, can be carried out in a variety of manners. The following describes various example approaches that may be implemented in connection with these figures and/or other embodiments. Clones are randomly selected from the genome at a relatively high coverage Cov_{G} ranging from 7.5x to 10.0x, resulting in a high degree of overlap between clones in long contiguous regions; for Cov_{G}=7.5 with clones of size 150 Kb, clone contigs (e.g., or contiguously overlapping sets of clones) of roughly 36 Mb are obtained. For Cov_{G}=10.0 or higher, clone contigs cover entire chromosomes. The clones are sequenced to relatively low coverage, and from their reads, a determination is made as to which sets of clones overlap and in which ordering they appear along the genome as discussed further below.

A clone graph G=({Oᵢ}, W) is constructed, in which nodes are clone contigs that are initialized to be sequences of one clone each: Oᵢ=<Cᵢ>; weighted edges connect the nodes with weight Wᵢⱼ equal to the count of unique k-mers shared between the two clones Cᵢ and Cⱼ. The value k=24 is used, which is large enough to isolate unique k-mers, and small enough to still be sensitive despite sequence errors. A unique k-mer is defined as one that appears at most three times the expected coverage level (3.0·Cov_{G}·Cov_{R}). The graph can be constructed efficiently by scanning through all the read data, and collecting each k-mer along with the clone that contains it in an array. The array is sorted by k-mers, and a determination is made as to how many and which clones contain each particular k-mer. Scanning through the array, the graph is constructed by accumulating counts to the edges for k-mers that satisfy the uniqueness constraint. For a graph with N_{N} nodes, N_{E}=N_{N}·2·(Cov_{G}−1) true edges between the nodes. To remove most spurious edges between non-overlapping clones, the N_{E} greatest edge weights are retained and the rest are discarded. As a reference example, for a 3 Gb mammalian genome with Cov_{G}=10.0, the size of the resulting graph is N_{N}=200,000 and N_{E}=3,600,000.

In applications in which every k-mer cannot be recorded in memory (e.g., applications involving large assemblies), a prime number p is selected to be large enough so that K/p k-mers can be stored in memory, where K is the total number of k-mers in all the read data. Each k-mer n is represented as a base-4 number, and (n mod p) is used to split the k-mer content into p roughly equally-sized classes. The genome is scanned p times (which is readily parallelizable), and all the graphs they produce are superimposed. In order to further reduce the computation time, a subset of the p jobs is selected and the edge weights are extrapolated.

With the graph G=({Oᵢ}, W), a greedy algorithm is applied to contract edges and order the clones within the nodes that are being merged. For each node Oᵢ, an ordered array of the clones <C₁ C₂ . . . Cₙ> that belong to the node are tracked, which initially is a single clone (e.g., consistent with the above). The algorithm goes through each edge Wᵢⱼ in the graph in order of decreasing weight. If the edge still connects two different nodes then we check that the two clones Cᵢ and Cⱼ are both “near the end” of their containing clone orderings, meaning that they are located within 3 clones of either end of their clone orderings. If this condition is satisfied, the two nodes are merged, their clone orderings are concatenated, and a small set of at most 7 clones around the junction is recorded. In some applications, the reordering is carried out by finding a permutation that best optimizes a scoring criterion that promotes orderings for which edge weights from a particular clone C to nearby clones increase as we move toward C:

$$Score ( 〈 {C}_{1} … {C}_{i} … {C}_{j} … {C}_{n} 〉 , i , j ) = {\sum }_{k = i}^{j} [ {\sum }_{m = 2}^{k - 1} s ( {C}_{k} , {C}_{m - 1} , {C}_{m} ) + {\sum }_{m = k + 1}^{n - 1} s ( {C}_{k} , {C}_{m + 1} , {C}_{m} ) ] \\ s ( {C}_{k} , {C}_{a} , {C}_{b} ) = \{ \begin{matrix}( {W}_{k , b} - {W}_{k , a} ) , & if {W}_{k , b} \geq {W}_{k , a} \\ - 4 ( {W}_{k , a} - {W}_{k , b} ) , & if {W}_{k , b} < {W}_{k , a}\end{matrix}$$

This scoring function considers the ordering of clones Cᵢ to Cⱼ, rewarding clone orders for which the weights increase along the ordering toward any particular clone, and heavily penalizing clone orders for which the weights are out of order. Neighboring clones share more k-mers and therefore their edges are given a higher weight. Optimization is done by searching the 7 factorial (7!) permutations. Neighbors or near-neighbors are generally joined first; as such, reordering is limited for many applications to no more than about 7 clones around a junction.

Once all of the edges are processed once in order of decreasing weight, many of which do not satisfy the conditions required for contraction, each of the remaining nodes represents a separate clone contig of ordered clones. Each clone contig is then assembled independently and in parallel.

Overlap detection and error correction is carried out as follows. The clone orderings are used to limit the computation of read overlaps. For each read in a particular clone Cᵢ, overlaps with other reads in clone Cᵢ or in other clones Cⱼ belonging to the same clone contig are considered, such that the two clones overlap and are nearby. Using clone orderings, the term W_{i,j}=0 is set for nodes that are in different clone contigs or too far apart. Alignments are seeded using exact 16-mers and a high error-cutoff threshold in order for overlap detection to be sensitive. High sensitivity is useful, for example, to identify likely repeats in the error correction stage; reads with too many errors are discarded. As read overlaps are detected, read overlap sets are constructed as [symbol] ={R_{q}|Rₚ and R_{q} align and extend each other}.

In some embodiments, the following error correction algorithm is applied in three separate passes over all the reads. In the first pass, the set of overlaps is augmented by looking for transitive read overlaps. For each read overlap set [symbol] , a multialignment of all the reads is created and any reads that do not pass the error-rate and correlation tests (described below) are screened out to produce a filtered read overlap set [symbol] ⊂ [symbol] . Each pair of reads in the new set Rₚ, R_{q}ε [symbol] is analyzed, and if an overlap is implied by their alignment through Rᵣ, reads Rₚ and R_{q} are inserted into their opposing read overlap sets [symbol] and [symbol] .

The error-rate test filters out reads having a number of differences from the majority that exceeds three times the expected error rate. The correlation test examines each read and at every column for which it disagrees from the majority of overlapping reads, counts the number of other reads that agree with it. If this number exceeds a heuristic threshold, the read is marked as correlated. The correlation test thus filters out repeat-induced overlaps.

Errors in the homopolymeric run count are handled separately; these are generally correlated even when there are no false overlaps. For example, in a homopolymeric run count of 20, several reads may exhibit run counts of 19 or 21. Errors are identified and screened out, and the correlation test is then applied, by counting the run lengths in all the reads in the multiple alignment and ignoring differences in run counts that fall within a small threshold of the average run count.

In the second pass through the reads, an augmented set of overlaps is used to better identify false overlaps using the correlation test. For each read Rᵣ, multiple alignments are constructed from the new set of reads in [symbol] . The error-rate and correlation tests are applied to the reads and those that fail are removed from [symbol] .

In the third pass, the resulting highly specific read overlap sets are used to construct multiple alignments that are used to correct errors. A simple majority vote is used to determine the consensus character for each column. At this point, errors in the run count are corrected by computing the average run count for each homopolymeric run and modifying those that differ by a small amount. Corrections cumulatively influence further error-correction alignments.

Referring back to FIG. 2, and as may also be applicable to FIG. 3, contig assembly at 200, 210 and 220 can be carried out in a variety of manners. The following describes various example approaches that may be implemented in connection with these figures and/or other embodiments.

Using pre-processing steps as described above, resulting clones have been ordered, read overlaps have been computed and reads have been corrected so that most overlaps are generally error-free. Three stages of assembly are applied (i.e., as may be implemented respectively at 200, 210 and 220 in FIG. 2). Each stage constructs longer contigs that cover progressively larger windows of the genome. In the first stage, read sets, which consist of sets of reads that are localized within small subregions of each clone, are created and assembled independently. The resulting first-stage contigs are combined in larger contig sets that collect all contigs contained within each clone in a second stage. The resulting second-stage contigs are merged into one final assembly per clone contig in a third stage. These three stages are described in greater detail as follows, in connection with figures and related discussion.

The clones are selected at high coverage and each clone is sequenced to a low coverage Cov_{R} between 1.5x and 2.0x. To obtain full coverage, the reads are combined from multiple overlapping clones. Clone ordering is used to isolate the locations of individual reads to windows much smaller than a clone length (e.g., dramatically reducing the copy number of each repeat within a short region to the minimum and bypassing the notoriously difficult “repeat resolution” problem in fragment assembly at this stage).

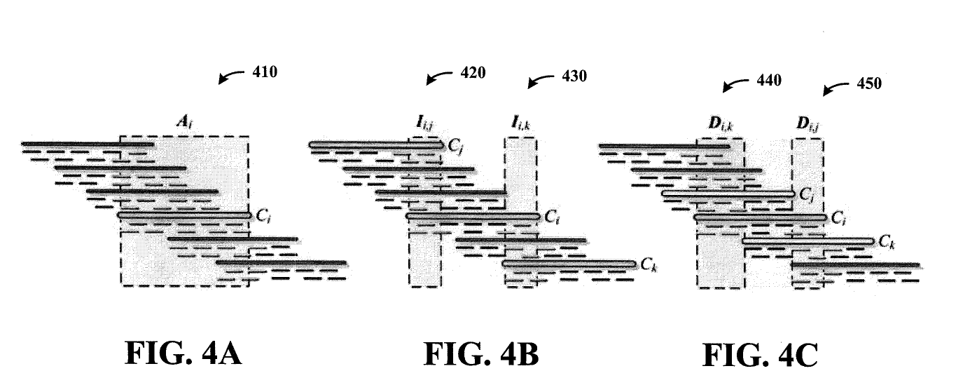

Referring to FIG. 4A, FIG. 4B and FIG. 4C, three types of localized read sets are constructed in connection with a first stage as referenced above, in accordance with various example embodiments of the present invention. FIG. 4A shows the construction of clone read sets, FIG. 4B shows the construction of intersection read sets, and FIG. 4C shows the construction of difference read sets. These read sets include all reads that putatively overlap a region of the genome delineated by clone extent endpoints.

The clone read sets Aᵢ (410) in FIG. 4A are constructed by first defining the clone extent of each read, which is the inferred set of clones spanning the location of the read in the genome, and then for every clone Cᵢ, all reads that contain Cᵢ in their clone extent are collected. In this regard, a clone read set is created for each clone, with each read set including all the reads overlapping the clone (including reads from other clones).

In FIG. 4B, Intersection read sets I_{i,j} and I_{i,k} (420, 430) are constructed by finding, for Cᵢ, the clones Cⱼ and Cₖ that overlap Cᵢ minimally to the left and right, and intersecting their respective inferred clone read sets. That is, pairs of clones that have small overlaps are identified and their clone read sets are intersected to obtain a set of reads spanning the overlap region.

In FIG. 4C, difference read sets D_{i,j} and D_{i,k} (440, 450) are constructed similarly by finding, for Cᵢ, the clones Cⱼ and Cₖ that overlap it maximally to the left and right, and subtracting the respective inferred clone read sets. Each read set is assembled independently (i.e., using the Euler assembler). Thus, the difference read sets are created by finding pairs of clones that have large overlaps and subtracting their clone read sets to obtain a set of reads spanning the region of the genome covered by one clone and not the other.

Prior to constructing the above read sets, the clone extent Eᵣ is computed of every read Rᵣ, which is defined as the set of clones that overlap the read Rᵣ. Initially, each clone extent is empty. If C(Rᵣ) denotes the source clone for read Rᵣ, then for every read-pair overlap (Rₚ, R_{q}), the source clones are inserted into the opposing clone extent (i.e., C(Rₚ) is inserted into E_{q} and C(R_{q}) is inserted into Eₚ).

Since each clone is covered by reads to low depth, a given read may have clones that span it but do not contain any overlapping reads. In some embodiments, transitive overlaps are used to improve the sensitivity of placing clones within the clone extent sets by iteratively applying the following procedure. The next set of clone extents {E′ᵣ} is constructed by first setting them equal to {Eᵣ}. For each read pair overlap (Rₚ, R_{q}), E′_{q}←E′_{q}∪Eₚ and E′ₚ←E′ₚ∪E_{q} are set. Although this process creates false clone-read overlaps, in practice lower specificity of the clone extents is less likely to create misassemblies than lower sensitivity. Very high sensitivity with little loss of specificity for the clone read sets and intersection read sets can be achieved by iterating this procedure twice for 20.0x coverage and four times for 11.25x coverage. The clone ordering is used to infer any missing intervening clones; given clone ordering <C₁ C₂ . . . Cₙ>, for read Rr, the minimum 1≦i≦n s.t. Cᵢ∈Eᵣ and maximum 1≦j≦n s.t. Cⱼ∈Eᵣ. Then, Eᵣ is set as Eᵣ={Cᵢ Ci+1 . . . Cⱼ}.

The clone read sets {Aᵢ} are constructed by setting Aᵢ={Rᵣ|Cᵢ∈Eᵣ}. The intersection read sets {I_{i,j}} and {I_{i,k}} are created, two per clone Cᵢ, by finding the minimally overlapping clone Cⱼ to the left in the clone ordering, j=argmin_{j<i} W_{i,j}, as well as the minimally overlapping clone Cₖ to the right in the clone ordering, k=argmin_{k>i} W_{i,k}. Two intersection sets I_{j,i}=Aⱼ∩Aᵢ and I_{i,k}=Aᵢ∩Aₖ are then constructed. The difference read sets {D_{i,j}} and {D_{i,k}} are similarly created from each clone Cᵢ by finding the maximally overlapping clone Cⱼ to the left, j=argmax_{j<i} Wᵢⱼ, and Cₖ to the right, k=argmax_{k>i} Wᵢₖ, and then constructing D_{i,j}=Aᵢ−Aⱼ and D_{i,k}=Aᵢ−Aₖ.

Each of the read sets {Aᵢ}, {I_{j,k}}, and {D_{j,k}} are then assembled using Euler, which is perhaps the most accurate assembler because it will not merge overlaps for which there is ambiguity. Each assembly is computed independently and in parallel, resulting in sets of contigs {A′ᵢ}, {I′_{j,k}}, and {D′_{j,k}}.

Once read sets have been identified in a first stage as described above, contigs from the first stage are combined in larger regions to create contig sets in a second stage. A contig set Bᵢ is created for each clone Cᵢ. The contig set includes all the contigs from a read set that is completely contained within the extent of the clone Cᵢ. Given a read set, a determination is made as to whether its contigs belong to Bᵢ as follows. For clone read sets, the contigs in A′ᵢ are inserted only in Bᵢ. For intersection read sets (given an intersection read set I_{j,k}), the clones Cⱼ and Cₖ both contain the region intersected by Cⱼ and Cₖ, and so does every intervening clone Cᵢ in the clone ordering < . . . Cⱼ . . . Cᵢ . . . Cₖ . . . >. Therefore, the contigs in I′_{j,k} are inserted to Cⱼ, Cⱼ₊₁, . . . , Cₖ. For difference read sets (given a difference read set D_{j,k} where j<k (in other words, the clone Cₖ is being subtracted from the right end of clone Cⱼ)), any clone Cᵢ that is to the left of Cⱼ (i.e. < . . . Cᵢ . . . Cⱼ . . . Cₖ . . . >) and that has an overlap with Cₖ (W_{i,k}>0) is completely contained in Cᵢ. The difference sets D_{j,}k for which j>k have similar containers.

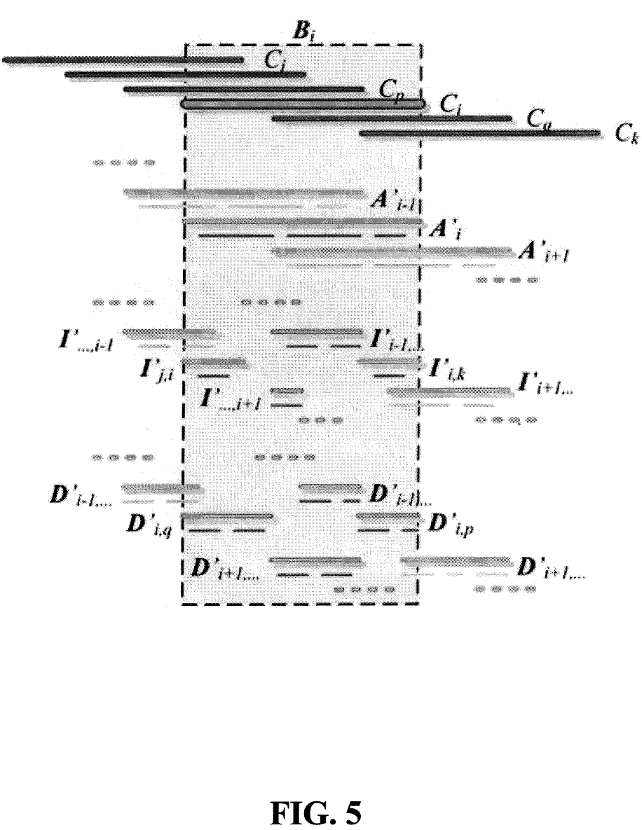

FIG. 5 shows an approach for the construction of contig sets from read set assemblies in a second stage as discussed in the preceding paragraph, according to another example embodiment of the present invention. For each clone Cᵢ, a contig set Bᵢ (500) is constructed by collecting all contig sets A′ᵢ, I′_{j,k}, and D′_{j,k} that logically should be contained completely within the span of the clone. With the above approaches and as shown in FIG. 5, contig sets can be computed as follows:

Bᵢ=A′ᵢ∪{I′_{j,k}|j≦i≦k}∪{D′_{j,k}|i≦j<k and W_{i,k}>0}∪{D′_{j,k}|k<j≦i and W_{i,k}>0}

Each contig set Bᵢ is assembled independently and in parallel (e.g., using Euler) to produce a set of even larger contigs B′ᵢ.

After assembly in the second stage, clone contigs are merged in a third stage as follows. The contigs are merged along entire clone contigs using an assembler that uses the clone ordering and clone overlap information to facilitate desirable memory usage as well as reduce the number of potential overlaps examined. The assembler considers each clone Cᵢ in a left-to-right fashion along a clone ordering, reading in all contigs that may overlap Cᵢ, which are the contigs from B′ᵢ and any other B′ⱼ for which there is an overlap W_{i,j}>0.

After finding all possible overlaps between the contigs under consideration, contigs for which there is no overlap ambiguity are merged. That is, if contig a is minimally extended to the right by contig b and then contig c, contigs b and c must also align with each other. This constraint avoids misassemblies.

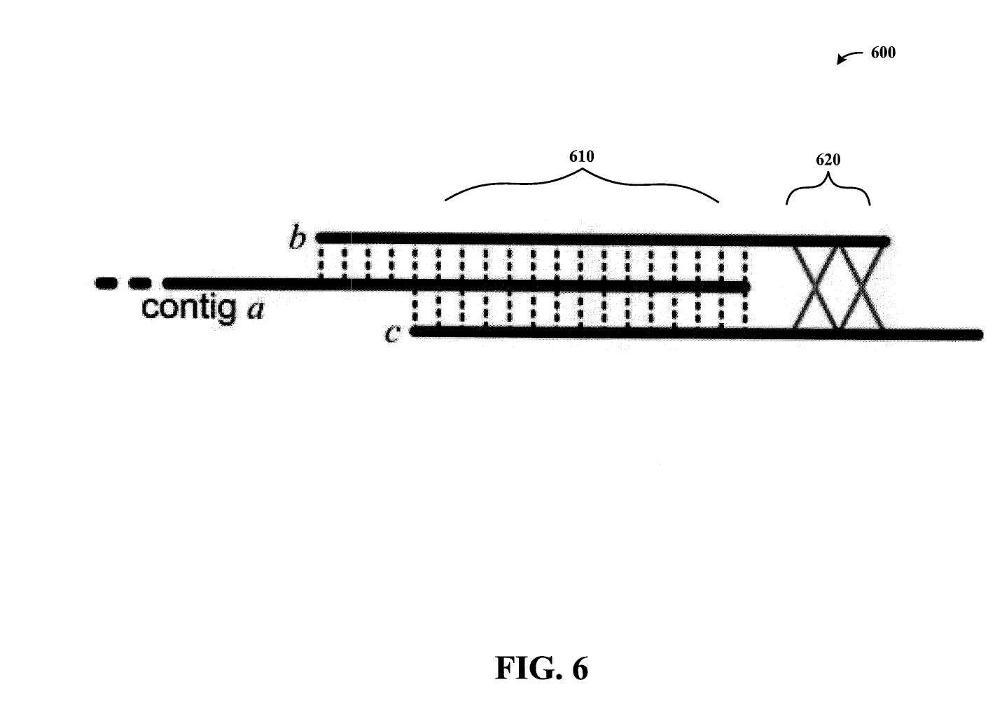

In connection with this third stage and merging approach, FIG. 6 shows an example approach 600 to merging contigs, according to another example embodiment of the present invention. The shown example overlapping condition involves contigs a, b and c that overlap at 610. However, contigs b and c do not fully overlap each other at 620, indicating a region of ambiguity such as a repeat boundary or misassembly. Where such an ambiguity exists, the contigs are not merged.

In some applications, heuristics are employed to find likely misassemblies by comparing contigs against themselves and other contigs and looking for suspiciously long, perfect overlaps that do not extend to the end. For each contig, a set of clones is kept, which is initially set to a single clone Cᵢ that corresponds to the contig set B′ᵢ of origin. As contigs are merged, the union of the sets of clones are taken. With this approach, conditions are detected wherein a particular contig will no longer overlap any clones under consideration; at such a point, the consensus sequence is formed. In certain implementations involving a CPU executing media-stored program instructions (e.g., electronically stored in CPU internal or downloadable program memory), the program instructions are executed by the CPU to perform steps corresponding to methods embodying principles of the present invention, and the consensus sequence is generated for further use (e.g., displayed and/or stored electronically).

The resulting assembly lists the contigs in a rough ordering along the clone contigs, but may not strictly order or orient them in scaffolds. For certain applications, scaffolding is carried out on the contigs using very light, paired reads as described above.

### Experimental Data and Related Embodiments

Various embodiments have been specifically implemented on a nonlimiting, experimental basis. Details of these embodiments have been published. For further information regarding details of these experimental embodiments, reference may be made to the article by Sundquist et al., Whole-Genome Sequencing and Assembly with High-Throughput, Short-Read Technologies, PLoS ONE (www.PLOSONE.org), May 30, 2007. This article, with its specifically disclosed embodiments which relate to embodiments disclosed infra, is incorporated by reference in its entirety (and attached hereto as an Appendix).

The various embodiments described above are provided by way of illustration only and should not be construed to limit the invention. Based upon the above discussion and illustrations, those skilled in the art will readily recognize that various modifications and changes may be made to the present invention without strictly following the exemplary embodiments and applications illustrated and described herein. Such modifications and changes do not depart from the true spirit and scope of the present invention, including that set forth in the following claims.

What is claimed is:

1. A method for genome sequencing, the method comprising:
as a random function, selecting a subset of fragments of a target genome;
replicating each fragment into clones;
ordering the clones into clone contigs based on sets of overlapping clones;
determining potential read overlaps from clone read data and validating base pairs of each read;
reading local assemblies of contigs from regions smaller than a clone length and assembling the local assemblies into read sets;
combining the assembled read sets into clone-sized regions; and
assembling the clone-sized regions into clone contigs.

2. The method of claim 1, further including tagging the cloned fragments with clone IDs, and using the clone IDs to identify a clone from which the read sets originate.

3. The method of claim 1, further including the step of identifying a clone from which the read sets originate based on uniquely tagged cloned fragments.

4. The method of claim 1, wherein reading local assemblies of contigs from regions smaller than a clone length includes:
finding all reads that overlap each particular clone,
performing intersection and subtraction operations on the sets of reads to isolate smaller regions, and
independently assembling each read set.

5. The method of claim 1, wherein selecting a subset of fragments of a target genome includes selecting a subset of fragments that cover the genome at high redundancy of at least about 4.0x coverage.

6. The method of claim 1, wherein selecting a subset of fragments of a target genome includes selecting a subset of fragments that cover the genome at high redundancy of at least about 4.0x coverage, and further including
acquiring sequencing reads from fragments at redundancy that is lower than said high redundancy, and
constructing the read sets by combining sequencing reads acquired from different overlapping fragments and assembling into local assemblies.

7. The method of claim 1, wherein validating includes
comparing overlapping read data from the sequence to detect overlapping reads that are different for common data, and
performing error correction on the overlapping reads that are detected as being different.

8. The method of claim 1, wherein validating includes detecting overlapping reads that are different for common data and performing error correction on the overlapping reads that are detected as being different.

9. A method for genome sequencing that uses validated clones generated from a subset of fragments of a target genome and ordered into clone contigs based on sets of overlapping clones, the method comprising:
reading local assemblies of contigs from validated clone regions smaller than a clone length and assembling the local assemblies into read sets;
combining the assembled read sets into clone-sized regions; and
assembling the clone-sized regions into clone contigs.

10. The method of claim 9, further including the step of providing the validated clones by detecting overlapping reads that are different for common data and performing error correction on the overlapping reads that are detected as being different.

11. The method of claim 9, wherein the step of assembling the clone-sized regions into clone contigs includes assembling the clone-sized regions into an entire clone contig.

12. The method of claim 9, wherein the step of assembling the clone-sized regions into clone contigs includes assembling the clone-sized regions for a genome.

13. The method of claim 9, further including the step of providing the validated clones and wherein validating includes
comparing overlapping read data from the sequence to detect overlapping reads that are different for common data, and
performing error correction on the overlapping reads that are detected as being different.

14. The method of claim 9, wherein reading local assemblies of contigs from regions smaller than a clone length includes:
finding all reads that overlap each particular clone,
performing intersection and subtraction operations on the sets of reads to isolate smaller regions, and
independently assembling each read set.

15. The method of claim 14, wherein each read includes raw data read from the sequence

16. A storage device comprising data representing computer-executable instructions that, in response to being accessed and executed by a computer, cause performance of a method for genome sequencing that uses validated clones generated from a subset of fragments of a target genome and ordered into clone contigs based on sets of overlapping clones, the method including the steps of:
reading local assemblies of contigs from validated clone regions smaller than a clone length and assembling the local assemblies into read sets;
combining the assembled read sets into clone-sized regions; and
assembling the clone-sized regions into clone contigs.


SEQUENCE LISTING (4 sequences)

SEQ ID NO: 1 - DNA, 19 bp, Artificial Sequence
  Synthetic construct
agcagtgatcgatgattac

SEQ ID NO: 2 - DNA, 19 bp, Artificial Sequence
  Synthetic construct
atgctcagacttagctatt

SEQ ID NO: 3 - DNA, 19 bp, Artificial Sequence
  Synthetic construct
gcaatttatatcagagaca

SEQ ID NO: 4 - DNA, 19 bp, Artificial Sequence
  Synthetic construct
agcagaatcgagagcaaga